(19) United States
(12) Patent Application Publication
(10) Pub. No.: US 2004/0063835 A1
(43) Pub. Date: Apr. 1, 2004

(54) Water-dilutable oils

(76) Inventors: Willibald Paar, Graz (AT); Maximilian Friedl, Gratkorn (AT); Michael Gobec, Graz (AT)

Correspondence Address: ProPat, L.L.C., 2912 Crosby Road, Charlotte, NC, 28211-2815, US

(21) Appl. No.: 10/618,789

(22) Filed: Jul. 14, 2003

(30) Foreign Application Priority Data
Jul. 19, 2002 (AT) A 1101/2002

(51) Int. Cl. C08K 5/09; C07C 53/00

(52) U.S. Cl. 524/313; 524/284; 562/887

(57) ABSTRACT

Water-dilutable oils ABC comprising building blocks derived from oils A having on average at least one olefinic double bond per molecule, cyclic olefinically unsaturated anhydrides B, and organic acids C which contain at least one acid group and at least one further group which reacts with a cyclic acid anhydride with ring opening and addition, a process for their preparation, and their use as an addition to aqueous resins and dispersions

### FIELD OF THE INVENTION

The invention relates to water-dilutable oils.

### BACKGROUND OF THE INVENTION

Water-dilutable oils have already been known from the prior art. They are prepared by reacting oils containing on average at least one olefinic double bond in the molecule with maleic anhydride. The anhydride groups are opened by reaction with water or alcohols to give the dicarboxylic acid or the monoester, and after neutralization with basic compounds the reaction products can be dispersed in water to form stable dispersions.

A disadvantage of these known products is that they lead only to dispersions with a limited oil content. The dispersions can be used as an admixture component to aqueous resins in order to enhance the gloss of the coatings produced with said resins. A further disadvantage of these products is that if dispersions prepared in this way are mixed with water-diluted acrylic resins or dispersions of other resins it is not uncommon to observe phase separation. This limits the technical usefulness and possible application of such dispersions.

### SUMMARY OF THE INVENTION

One object is therefore to provide water-dilutable oils which exhibit improved compatibility with dispersions of other water-diluted resins and lead to dispersions having relatively high oil contents.

This object is achieved by reaction products ABC of oils A with cyclic, olefinically unsaturated anhydrides B and organic acids C additionally containing groups which react with a cyclic acid anhydride by ring opening and addition.

The invention accordingly provides water-dilutable oils ABC comprising building blocks derived from oils A having on average at least one olefinic double bond per molecule, cyclic olefinically unsaturated anhydrides B, and organic acids C which contain at least one acid group and at least one further group which reacts with a cyclic acid anhydride by ring opening and addition.

A water-dilutable substance here is one which on dilution with water does not undergo spontaneous macroscopic separation.

The invention further provides a process for preparing the products ABC by first reacting oils A with the cyclic olefinically unsaturated anhydrides B. In the second stage the anhydride groups are then consumed by reaction with the acids C, forming one acid group attached directly to the carbon chain of the oil and one further such group, which as a result of the residue of the functional acid C, which acts as a spacer, is at a relatively large distance from the carbon chain. It is assumed that this steric difference produces the improved water compatibility.

It is likewise possible in the first stage, by reacting the acid C with the cyclic anhydride B, to produce an intermediate BC which can then be reacted with the oil A.

A further possibility is to perform the reaction by reacting all of the components together.

### DETAILED DESCRIPTION OF THE PREFERRED EMBODIMENTS

The oils A contain on average at least one olefinic double bond per molecule. It is possible to use both natural oils (esters of glycerol with fatty acids) and synthetic oils, which can be obtained by oligomerization or polymerization of preferably polyunsaturated olefins. Particularly suitable oils are drying and semidrying oils, examples being vegetable oils such as linseed oil, soybean oil, rapeseed oil, colza oil, cottonseed oil, peanut oil, tall oil, sunflower oil, safflower oil, and jojoba oil, animal oils such as whale oil and fish oil, and synthetic oils such as polybutadiene oil or other oils based on α,ω-diolefins and also oils of copolymers of α,ω-diolefins and monoolefins. Particularly preferred oils are those having an iodine number of at least 90 g/g, in particular from 100 to 200 g/g.

Particularly suitable cyclic unsaturated anhydrides B are maleic anhydride, citraconic anhydride, itaconic anhydride, dodecenylsuccinic anhydride, and tetrahydrophthalic anhydride. Maleic anhydride is particularly preferred.

Suitable acids C include organic acids having at least one acid group and at least one group which is reactive with respect to acid anhydrides by ring opening and addition. The acid group is preferably selected from carboxyl groups, phosphonic acid groups, and sulfonic acid groups. The reactive groups may be hydroxyl, amino, and mercaptan groups. Particular preference is given to aliphatic hydroxycarboxylic acids having from 2 to 20 carbon atoms. It is also possible to use epoxidized acids provided a stoichiometrically sufficient amount of water is present. Preference is given to hydroxy acids such as glycolic acid, lactic acid, γ-hydroxybutyric acid, δ-hydroxyvaleric acid, ε-hydroxycaproic acid, and also polyhydroxy acids such as dimethylolpropionic acid, tartaric acid, and uvic acid. Suitable amino acids include for example β-alanine, γ-aminobutyric acid, δ-aminovaleric acid, and ε-aminocaproic acid. Suitable mercapto acids are mercaptoacetic acid, 3-mercaptopropionic acid, 2-mercaptoethanesulfonic acid, 3-mercapto-1-propanesulfonic acid, 2,3-dimercapto-1-propanesulfonic acid, mercaptosuccinic acid, 2,3-dimercaptosuccinic acid, and mercaptopyruvic acid.

Among the phosphonic acids mention may be made of 3-aminopropylphosphonic acid and 2-aminoethylphosphonic acid.

It is likewise possible to use the more strongly acidic sulfonic acids, especially 4-amino-1-naphthalenesulfonic acid, 4-amino-3-hydroxy-1-naphthalenesulfonic acid, aminomethanesulfonic acid, 4-hydroxy-1-naphthalenesulfonic acid, and 3-amino-1-propanesulfonic acid.

The invention finally also provides for the use of the water-dilutable oils as an addition to aqueous dispersions of other resins, and also to the water-dilutable binders thus prepared which comprise the water-dilutable oils ABC. The water-dilutable oils of the invention can be used with particular advantage as an addition to aqueous acrylic resins or polyurethane resin dispersions, in which case the mass ratio of oil to resin can be from 1:19 to 1:4. A particular advantage of such mixtures is that the drying behavior of the hybrid binder can be tailored precisely to the particular end use through the mixture.

By aqueous acrylic resins are meant here aqueous dispersions of copolymers containing at least a mass fraction of 20% of building blocks derived from acrylic or methacrylic compounds. Such acrylic or methacrylic compounds are preferably esters of acrylic or methacrylic acid with aliphatic, preferably monohydric or dihydric, alcohols having 1 or from 2 to 20 carbon atoms, the nitrites of these acids, and the acids themselves. As further constituents the copolymers contain in particular building blocks derived from the monomers styrene, vinyltoluene, vinyl chloride, vinyl esters such as vinyl acetate, vinyl propionate or vinyl esters of the Versatic acids.

Aqueous polyurethane resin dispersions are known and contain building blocks derived from polyhydroxypolyethers, polyhydroxypolyesters, polyhydroxypolycarbonates, polyfunctional isocyanates, if desired from low molecular mass polyols, and from polyhydroxy compounds having sterically hindered acid groups, such as dimethylolpropionic acid, for example, which following at least partial neutralization produce the required hydrophilicity of the resin.

Hybrid binders of this kind, which in addition to the water-dilutable oils also include at least one further water-dilutable binder selected in particular from acrylate copolymers and polyurethanes, are especially suitable for preparing water-dilutable coating materials for the coating of wood, paper, cardboard, and metals, and are used in particular in the coating of wood.

The examples which follow illustrate the invention. Concentrations stated in “%” are mass fractions of the dissolved substance in the solution (mass of the dissolved substance divided by the mass of the solution)

### EXAMPLES

### Example 1

### Synthesis of a Maleinized Acid-Modified Oil

880 g of linseed oil (1 mol) were reacted with 220 g (2.25 mol) of maleic anhydride at 210° C. until formation of the adduct was complete. After the reaction mixture had been cooled to 95° C., 235 g of lactic acid (80% strength solution in water, 2.25 mol) were added. About 50 g of water were distilled off at 100° C. under reduced pressure.

### Example 2

### Synthesis of a Maleinized Oil (Comparative)

Example 1 was repeated but replacing the lactic acid by water (10% excess over the stoichiometric amount) for the ring opening of the maleic anhydride groups.

### Example 3

### Compatibility Test

The oils from Examples 1 and 2 were mixed with a commercially available acrylate dispersion (based on styrene and acrylate, mass fraction of solids about 50%, viscosity according to ISO 2555 at 23° C., spindle 2: about 900 mPa·s, pH 8.5, ®Mowilith LDM 6621) in a mass ratio (based in each case on solids fraction) of 1:9. The mixtures were stored at 40° C. While the mixture containing the oil from Example 2 had separated after only one hour, the mixture with the oil from Example 1 remained stable for 8 weeks (after which observation was terminated).

### Example 4

### Preparation of Water-Dilutable Coating Materials

The modified oil from Example 1 was blended with the dispersion described in Example 3 to give a binder, and with the further additions according to the formula the paint 2 was formulated. For this purpose the binder mixture was introduced initially, followed by slow stirring of the pigment paste and the additives, in portions, in the order stated, into the initial charge. The paint was then adjusted with water to the desired viscosity of 200 mPa·s (measured in accordance with ASTM-D 4287-88, the so-called “high shear” method, at 23° C. and 10 000 s⁻¹). For comparison (paint 1) the unmodified dispersion was used (the same formula but without addition of oil). By adding 25% strength aqueous ammonia solution the pH was adjusted to 8.5.

Formula for 100 g of paint:

| | | |
|---|---|---|
| 51.70 | g | ® Mowilith LDM 6621 |
| 7.40 | g | modified oil from Example 1 |
| 27.30 | g | pigment paste*) |
| 0.20 | g | aqueous ammonia solution (25 g NH₃ in 100 g |
| | | solution |
| 0.20 | g | ® Additol VXW 6386 (defoamer, Solutia Austria GmbH) |
| 1.50 | g | ® Mowilith VDM 7000 (rheology additive, |
| | | acrylate thickener, Clariant AG) |
| 0.20 | g | ® BYK 346 (substrate wetting agent, Byk Chemie) |
| 2.50 | g | propylene glycol |
| 9.00 | g | fully deionized water |

*Composition for 100 g of pigment paste:

| | | |
|---|---|---|
| 24.8 | g | fully deionized water |
| 2.9 | g | ® Additol VXW 6200 (dispersant, Solutia |
| | | Austria GmbH) |
| 0.7 | g | ® AMP 90 (2-amino-2-methyl-1- |
| | | propanol, Angus Chemie) |
| 0.7 | g | ® Agitan 295 (mineral oil-based |
| | | defoamer, Münzing Chemie GmbH) |
| 70.9 | g | titanium dioxide pigment (® Kronos 2310, Kronos |
| | | Titan GmbH). |

The substances were mixed in the stated order and then dispersed in a bead mill for about 40 minutes.

The paints were found to have the following measured data:

| | |
|---|---|
| Paint solids (mass fraction of solids): | about 47% |
| paint viscosity, ASTM D 4287-88, | about 200 mPa · s |
| 10 000 s⁻¹ at 23° C.: | |
| tack-free time, 152 μm applicator: | about 30 min |
| flash point, DIN EN 22 719: | >100° C. |

### Example 5

### Production and Testing of Coatings

Paints 1 and 2 were applied to glass plates (150 μm applicator) and after air drying at room temperature (23° C.) for about 24 hours assessed for qualities including gloss and gloss haze. The results are compiled in Table 1 below:

| | Gloss (measurement | Gloss haze (value |
|---|---|---|
| | angle 20°) | range 10-500) |
| | DIN 67530 | ASTM E 430 |
| Paint 1 | 65 | 170 |
| Paint 2 | 80 | 150 |

From the measurements the improvement in the gloss and the reduction in the haze brought about by the addition of the oil modified in accordance with the invention are clearly evident.

What is claimed is:

1. A water-dilutable oil ABC comprising building blocks derived from oils A having on average at least one olefinic double bond per molecule, cyclic olefinically unsaturated anhydrides B, and organic acids C which contain at least one acid group and at least one further group which reacts with a cyclic acid anhydride by ring opening and addition.

2. The water-dilutable oil ABC as claimed in claim 1, wherein the oils A are selected from drying and semidrying oils having an iodine number of at least 90 g/g.

3. The water-dilutable oil ABC as claimed in claim 1, wherein the cyclic olefinically unsaturated anhydrides B are selected from maleic anhydride, citraconic anhydride, and itaconic anhydride, dodecenylsuccinic anhydride and tetrahydrophthalic anhydride.

4. The water-dilutable oil ABC as claimed in claim 1, wherein the organic acids C are selected from organic carboxylic, sulfonic, and phosphonic acids each containing at least one further reactive group selected from hydroxyl, amino, and mercaptan groups.

5. The water-dilutable oil ABC as claimed in claim 1, wherein the organic acids C are selected from aliphatic hydroxycarboxylic acids having from 2 to 20 carbon atoms.

6. A process for preparing water-dilutable oils ABC by reacting drying or semidrying oils A having on average at least one double bond per molecule, cyclic olefinically unsaturated anhydrides B, and organic acids C which contain at least one acid group and a further group which reacts with a cyclic acid anhydride with ring opening and addition.

7. The process as claimed in claim 6, wherein in the first step the oils A are reacted with the acid anhydrides B and this reaction product is reacted with the acid C.

8. The process as claimed in claim 6, wherein in the first step the acids C are reacted with the anhydride B to give an adduct BC which in the second step is reacted with the oil A.

9. A water-dilutable binder for coating materials, comprising the water-dilutable oil ABC as claimed in claim 1.

10. A water-dilutable coating material comprising the water-dilutable oil ABC as claimed in claim 1 and at least one further binder selected from aqueous dispersions of acrylate copolymers and of polyurethanes.
